(19) United States
(12) Patent Application Publication
(10) Pub. No.: US 2009/0318697 A1
(43) Pub. Date: Dec. 24, 2009

(54) PROCESS FOR PREPARING A PIPERAZINE DERIVATIVE

(75) Inventors: Iolanda Chamorro Gutiérrez, Santa Coloma de Farners (ES); Raül Xifra Garcia, Caldes de Malavella (ES)

Correspondence Address: LEYDIG VOIT & MAYER, LTD, TWO PRUDENTIAL PLAZA, SUITE 4900, 180 NORTH STETSON AVENUE, CHICAGO, IL, 60601-6731, US

(73) Assignee: Medichem, S.A., Sant Joan Despi (ES)

(21) Appl. No.: 12/487,854

(22) Filed: Jun. 19, 2009

Related U.S. Application Data
(60) Provisional application No. 61/074,036, filed on Jun. 19, 2008.

(51) Int. Cl. C07D 241/04

(52) U.S. Cl. 544/400

(57) ABSTRACT

Disclosed is a process for preparing purified ranolazine of formula (I), which is indicated for the chronic treatment of angina, comprising reacting 1-[(2,6-dimethylphenyl)aminocarbonyl]piperazine with 1-phenoxy-2,3-epoxypropane in an inert solvent followed by precipitating the ranolazine.

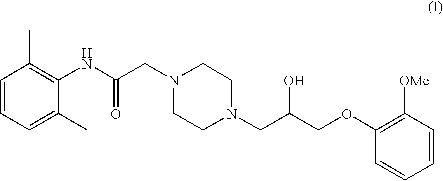

### CROSS-REFERENCE TO A RELATED APPLICATION

This patent application claims the benefit of U.S. Provisional Patent Application No. 61/074,036, filed on Jun. 19, 2008, which is incorporated by reference.

### BACKGROUND OF THE INVENTION

Ranolazine (compound of formula (I)) is the international common accepted name for (±)-1-[3-(2-methoxyphenoxy)-2-hydroxypropyl]-4-[N-(2,6-dimethylphenyl)carbamoylmethyl]piperazine, and has an empirical formula of C₂₄H₃₃N₃O₄ and a molecular weight of 427.54 g/mol.

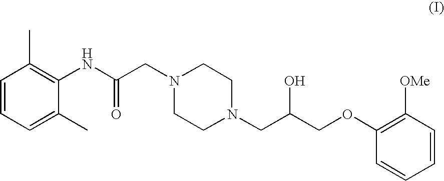

Ranolazine is an active pharmaceutical substance which has antianginal and anti-ischemic effects that do not depend upon reductions in heart rate or blood pressure. In the United States, ranolazine is marketed under the name RANEXA™, and is indicated for the treatment of chronic angina.

The preparation of ranolazine is disclosed in U.S. Pat. No. 4,567,264 (“the '264 patent”), which is incorporated herein by reference. The '264 patent discloses a preparation of ranolazine base by condensing N-(2,6-dimethylphenyl)-2-piperazin-1-ylacetamide (compound of formula (II)) with 2-[(2-methoxyphenoxy)-methyloxirane (compound of formula (III)), as depicted herein at Scheme 1.

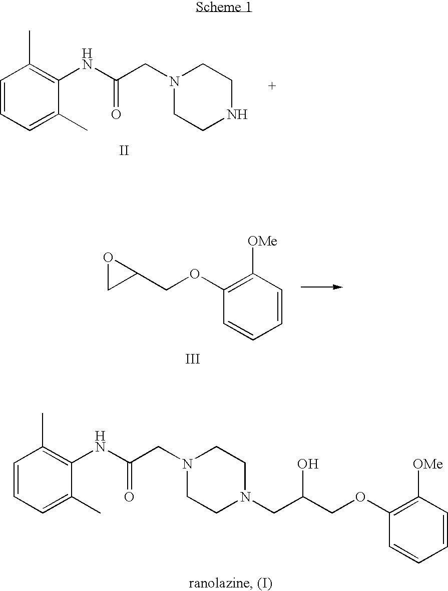

The method depicted in Scheme 1 suffers from one or more the following drawbacks: long reaction times, moderate yield, and/or tedious purification. For example, the reaction depicted in Scheme 1 is characterized by a reaction time of 5 hours, yields of about 59%, and/or the need for column chromatography to purify the product. Moreover, in the synthesis depicted in Scheme 1, is further characterized by the production and isolation of ranolazine dihydrochloride salt as a first product, and thus results in an additional synthetic step in order to obtain ranolazine.

The '264 patent also discloses a second preparation of ranolazine via condensation of [(2,6-dimethylphenyl)aminocarbonylmethyl]chloride (compound of formula IV) with 1-[3-(2-methoxyphenoxy)-2-hydroxy-propyl]piperazine (compound of formula V), as depicted herein at Scheme 2.

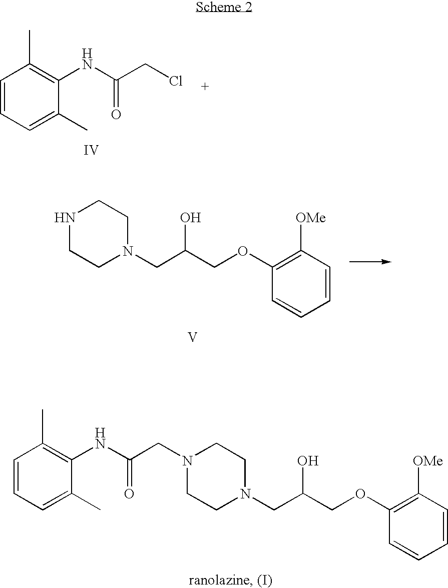

However, the synthesis of ranolazine depicted in Scheme 2 does not avoid the drawbacks of the synthesis of Scheme 1, and is also characterized by one or more of moderate yields, lengthy reaction times (i.e., more than 12 hours), purification using column chromatography, and the first solid isolated in the reaction is the dihydrochloride salt.

International Publication No. WO 2006/008753 (“the '753 publication”), also discloses preparing ranolazine using the method depicted in Scheme 2, but without the use of column chromatography. However, the method disclosed in the '753 publication is less than ideal for preparing ranolazine on a large scale. For example, lengthy reaction times are required (about 18 hours) and hazardous solvents such as dichloromethane and N,N-dimethylformamide are employed. Further, the isolation of ranolazine base comprises an acid base treatment of the reaction mixture, and therefore, proceeds via the dihydrochloride salt of ranolazine, although the dihydrochloride salt is not isolated.

In addition, no information about the purity of ranolazine product is provided, however, the crude ranolazine is purified through a second formation of its dihydrochloride salt and subsequent freeing of the base.

In view of the foregoing there is an umnet need for an industrially practicable process for preparing ranolazine in a pure form.

### BRIEF SUMMARY OF THE INVENTION

The present invention provides an improved process for preparing piperazine derivatives, including ranolazine of formula (I):

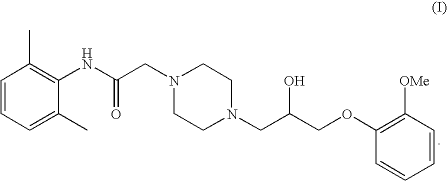

In an embodiment, the invention provides a process for preparing ranolazine comprising reacting reacting 1-[(2,6-dimethylphenyl)aminocarbonyl]piperazine of formula (II):

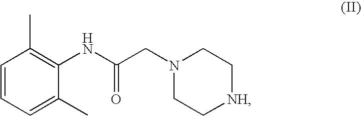

with 1-phenoxy-2,3-epoxypropane of formula (III):

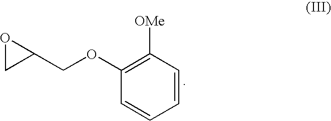

Processes of the invention are characterized by one or more of the following advantages: avoid using column chromatography, avoid lengthy reaction times, and reduce the use of hazardous solvents. Moreover, processes of the invention directly afford ranolazine free base as a pure, or substantially pure, solid which precipitates in the reaction medium, and is easily isolated and purified by means of simple filtration and slurrying, thereby avoiding one or more of the steps of formation, purification, and freeing of ranolazine dihydrochloride salt.

Solid ranolazine produced in accordance with processes of the invention has a high purity and a preferable particle size distribution.

### DETAILED DESCRIPTION OF THE INVENTION

In accordance with an embodiment, the invention provides a process for preparing solid ranolazine (compound of formula (I)):

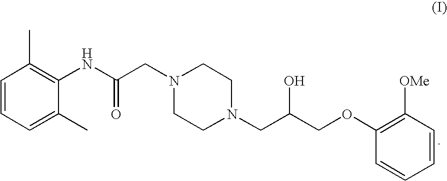

In keeping with an embodiment of the invention, a process for preparing solid ranolazine of formula (I) comprises i) reacting 1-[(2,6-dimethylphenyl)aminocarbonyl]piperazine of formula (II):

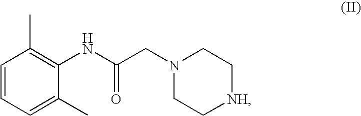

with 1-phenoxy-2,3-epoxypropane of formula (III):

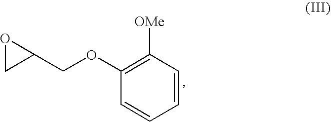

in an inert solvent to obtain a solution comprising ranolazine; ii) causing solid ranolazine to precipitate from the solution, to obtain a suspension; and iii) isolating solid ranolazine from the suspension.

In some embodiments, a process of the invention comprises iv) optionally, further purifying the solid ranolazine.

Applicants have surprisingly discovered that ranolazine base can be directly precipitated and isolated from the reaction medium to afford ranolazine base with acceptable purity, and hence without the need of preparing, purifying, and freeing the dihydrochloride salt, and/or using column chromatography purification. The ranolazine obtained by the process of the invention can be further purified to increase its chemical purity. In addition, the process of the invention requires short reaction times and reduces the use of hazardous solvents.

By way of example, illustrative inert solvents suitable for conducting the reaction of a compound of formula (II) with a compound of formula (III) in accordance with embodiments of the invention include alcohols, such as lower alcohols. In a preferred embodiment, the inert solvent of step i) comprises one or more C₁-C₆ alcohol solvents. In a more preferred embodiment, the C₁-C₆ alcohol solvent is isopropanol.

In an embodiment, the inert solvent is free of halogen or amide groups.

In an embodiment, the inert solvent is free of a Class 1 or Class 2 solvents as defined by the International Conference on Harmonisation of Technical Requirements for Registration of Pharmaceuticals for Human Use (ICH).

In some embodiments, a reaction mixture comprising a compound of formula (II), a compound of formula (III), and an inert solvent is heated to obtain a solution comprising ranolazine.

In keeping with embodiments of the invention, solid ranolazine precipitates from solution to form a suspension. In a preferred embodiment, a solution of ranolazine is cooled to cause solid ranolazine to precipitate from solution, thereby forming a suspension. In a preferred embodiment, a solution comprising ranolazine is cooled to a temperature of about 20-25° C. to induce the formation of a solid ranolazine precipitate.

In some embodiments, precipitation of solid ranolazine from solution is caused by effectively increasing the concentration of ranolazine in solution (e.g., removing solvent from the solution). In a preferred embodiment, the volume of inert solvent of a solution of ranolazine is reduced by distilling the solvent, thereby causing solid ranolazine to precipitate from solution and form a suspension.

In some embodiments, precipitation of solid ranolazine from solution is caused by effectively decreasing the solubility of ranolazine in solution. In a preferred embodiment, adding an anti-solvent to a solution of ranolazine causes solid ranolazine to precipitate from solution, thereby forming a suspension.

In keeping with embodiments of the invention, a suspension of solid ranolazine in an inert solvent can be obtained from a solution of ranolazine using any of the methods described herein to cause precipitation.

In keeping with embodiments of the invention, solid ranolazine is isolated from a suspension of ranolazine. In a preferred embodiment, solid ranolazine is isolated by filtering the suspension.

In some embodiments, solid ranolazine obtained in accordance with processes of the invention is further purified. In a preferred embodiment, solid ranolazine is purified by crystallizing solid ranolazine in an organic solvent.

By way of example, suitable solvents for crystallizing solid ranolazine include dialkyl ketones, e.g., lower alkyl ketones. In a preferred embodiment, ranolazine obtained by steps i)-iii) is purified by crystallization from methyl ethyl ketone (MEK).

In keeping with embodiments of the invention, solid ranolazine prepared according to processes of the invention has a high purity. Typically, solid ranolazine in accordance with the invention has a purity greater than 99.4% as measured by HPLC. In a preferred embodiment, solid ranolazine in accordance with the invention has a purity greater than 99.9% as measured by HPLC.

In keeping with embodiments of the invention, solid ranolazine prepared in accordance with a process of the invention and which has been purified (e.g., crystallized from an organic solvent such as, for example, MEK)) has a particle size distribution wherein approximately 10% of the total volume (D₁₀) is made of particles having a diameter below approximately 10 μm, approximately 50% of the total volume (D₅₀) is made of particles having a diameter below approximately 40 μm, and/or approximately 90% of the total volume (D₉₀) is made of particles having a diameter below approximately 80 μm.

The following examples further illustrate the invention but, of course, should not be construed as in any way limiting its scope.

General Experimental Conditions

HPLC method for determining purity.

The chromatographic determination was carried out on a Symmetry C₁₈, 5 μm, 250×4.6 mm I.D column using a two component mobile phase (A and B) at a temperature of 25° C.

Mobile phase A was prepared by mixing 200 mL of acetonitrile with 800 mL of pH 4.4 buffer, which was prepared from 0.92 g of ammonium acetate dissolved in 800 mL of water. The pH of the buffer solution was adjusted to 4.4 with glacial acetic acid. The mobile phase was mixed and filtered through 0.22 μm nylon filter under vacuum.

Mobile phase B was acetonitrile.

The chromatograph was equipped with a UV detector monitoring 230 nm. The flow rate was 1.0 mL per minute.

The chromatograph was programmed as follows: initial 0-2 min. isocratic 100% mobile phase A, 2-16 min. linear gradient to 93% mobile phase A, 16-24 min. isocratic 93% mobile phase A, 24-45 min. linear gradient to 80% mobile phase A, 45-62 min. isocratic 93% mobile phase A, 62-82 min. linear gradient to 100% mobile phase A and 82-95 min. equilibration with 100% mobile phase A.

The test samples were prepared by dissolving the appropriate amount of sample to obtain a concentration of 1.6 mg/mL in mobile phase. Sample size was 20 μL.

Particle size distribution method.

The particle size for ranolazine was measured in a Malvern light scattering particle size analyzer with a 2 milliwatt Helium/Neon laser and a Fourier Transform lens system to focus the scattered laser light onto a photosensitive detector. The sample was run with a 2.40 mm lens and a MS1 Small Volume Sample Dispersion Unit with a stirred cell.

Samples for analysis were prepared by dispersing a weighed amount of ranolazine (approximately 0.01 g) in 20 mL of dispersant. The suspension was sonicated for 10 seconds and delivered drop-wise to a previously filled and background-corrected measuring cell until the obscuration reached the desired level. The sample was measured as quickly as possible after stabilization of the obscuration.

For particle size characterization, the notation Dₓ means that approximately X % by volume of the particles have a diameter less than a specified diameter. Thus, for example, D₉₀≦10.00 μm means that approximately 90% of the particles by volume in a composition preferably have a diameter less than approximately 10.00 μm. The values of D₁₀, D₅₀ and D₉₀ were specifically listed, each one being the mean of the six values available for each characterization parameter.

### EXAMPLE 1

### Preparation of Ranolazine

This example demonstrates a process for preparing ranolazine in accordance with an embodiment of the invention.

1-[(2,6-dimethylphenyl)aminocarbonyl]piperazine (100.00 g, 404.32 mmol, HPLC purity 99.6%, m.p.=114.5-115.4° C.) and 79.10 g (438.96 mmol, HPLC purity 94.6%) of 1-phenoxy-2,3-epoxypropane was suspended in isopropanol (600 mL) under a nitrogen atmosphere. The mixture was heated slowly to reflux temperature (82-83° C.) and the solution was kept at reflux temperature for 3 hours.

The solution was then cooled to 20-25° C. Precipitation was observed upon cooling to a temperature of about 45-47° C. The reaction mass was stirred for one hour at room temperature. Finally, the suspension was filtered and the solid was washed twice with isopropanol (2×10 mL). Yield: 246.75 g of wet crude ranolazine (90%, 155.55 g of estimated dry mass). HPLC purity: 96.51%.

246.75 g of crude ranolazine (estimated dry mass: 155.55 g) was suspended in 695 mL of methyl ethyl ketone and the suspension was heated to 80° C. Immediately thereafter the solution was cooled to 10-15° C. and maintained at that temperature for 1 hour. The suspension was filtered, washed with methyl ethyl ketone (10 mL). 165.43 g of wet ranolazine (132.34 g of estimated dry mass) was obtained (purification yield: 85%). HPLC purity: 99.44%.

165.43 g of ranolazine (132.34 g of estimated dry mass) was suspended in methyl ethyl ketone (576.20 mL) and the suspension was heated to 80° C. The solution was cooled to 60-65° C., filtered to remove insolubles, and then cooled to 0-5° C. and kept at that temperature for 1 hour. The suspension was filtered, washed with methyl ethyl ketone (10 mL) and dried in vacuum oven at 50-60° C. till constant weight. 122.11 g of ranolazine base was obtained (purification yield: 92%, global yield 70%). HPLC purity: 99.91%.

The ranolazine obtained showed a particle size distribution wherein approximately 10% of the total volume (D₁₀) is made of particles having a diameter below approximately 10 μm, approximately 50% of the total volume (D₅₀) is made of particles having a diameter below approximately 40 μm and approximately 90% of the total volume (D₉₀) is made of particles having a diameter below approximately 80 μm.

### EXAMPLE 2

### Preparation of Ranolazine

This is another example demonstrating a process for preparing ranolazine in accordance with an embodiment of the invention.

1-[(2,6-dimethylphenyl)aminocarbonyl]piperazine (20 g, 80.86 mmol, HPLC purity 99.55%) and 13.84 g (76.80 mmol, HPLC purity 91.95%) of 1-phenoxy-2,3-epoxypropane was suspended in isopropanol (120 mL) under a nitrogen atmosphere. Then the mixture was heated slowly to reflux temperature (82-83° C.) and the solution was maintained at reflux temperature for 3 hours.

The solution was then cooled to 20-25° C. Precipitation was observed upon cooling to a temperature of about 30° C. The reaction mass was stirred at room temperature. Finally, the suspension was filtered and the solid was with isopropanol (2×10 mL). Yield: 45.16 g of wet crude ranolazine (91.4%, 31.61 g of estimated dry mass). HPLC purity: 98.84%.

43.76 g of crude ranolazine (estimated dry mass: 30.63 g) was suspended in 137.2 mL of methyl ethyl ketone and the suspension was heated to 80° C. Immediately thereafter the solution was cooled to 20-25° C. and maintained at that temperature for 1 hour. The suspension was filtered and washed with methyl ethyl ketone (10 mL). 31.57 g of wet ranolazine (24.86 g of estimated dry mass) was obtained (purification yield: 81.16%). HPLC purity: 99.76%.

30.56 g of ranolazine (24.07 g of estimated dry mass) was suspended in 103 mL of methyl ethyl ketone and the suspension was heated to 80° C. The suspension was cooled to 20-25° C. and maintained at that temperature for 1 hour. The suspension was filtered, washed with methyl ethyl ketone (10 mL) and dried in vacuum oven at 50-60° C. until a constant weight. 22.73 g of ranolazine base was obtained (purification yield: 94.4%, global yield 70%). HPLC purity: 99.91%.

All references, including publications, patent applications, and patents, cited herein are hereby incorporated by reference to the same extent as if each reference were individually and specifically indicated to be incorporated by reference and were set forth in its entirety herein.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A process for preparing solid ranolazine (compound of formula I), 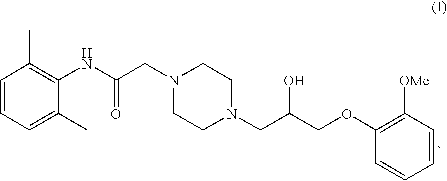
said process comprising:
i) reacting 1-[(2,6-dimethylphenyl)aminocarbonyl]piperazine of formula (II): 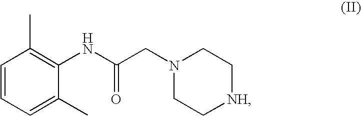
with 1-phenoxy-2,3-epoxypropane of formula (III): 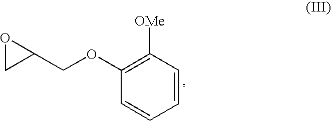
in an inert solvent to obtain a solution comprising ranolazine of formula (I);
ii) causing solid ranolazine to precipitate from the solution, to obtain a suspension;
iii) isolating solid ranolazine from the suspension; and
iv) optionally, purifying the solid ranolazine.

2. The process of claim 1, wherein the inert solvent comprises a C₁-C₆ alcohol solvent.

3. The process of claim 2, wherein the C₁-C₆ alcohol solvent is isopropanol.

4. The process of claim 1, wherein the reaction of step i) comprises heating the reaction mixture.

5. The process of claim 1, wherein step ii) comprises cooling the solution of step i).

6. The process of claim 5, wherein the cooling comprises cooling to at least 25° C.

7. The process of claim 1, wherein step ii) comprises reducing the volume of the inert solvent by distilling the solvent.

8. The process of claim 1, wherein step ii) comprises adding an anti-solvent to the solution.

9. The process of claim 1, wherein step iii) comprises filtering the suspension.

10. Solid ranolazine obtained according to the process of claim 1.

11. The process of claim 1, wherein step iv) comprises crystallizing solid ranolazine in an organic solvent.

12. The process of claim 11, wherein the organic solvent is methyl ethyl ketone.

13. Solid ranolazine obtained according to the process of claim 11.

14. The solid ranolazine of claim 13, wherein the solid ranolazine has a purity greater than 99.4% as measured by HPLC.

15. The solid ranolazine of claim 13 having a particle size distribution wherein approximately 10% of the total volume (D₁₀) is made of particles having a diameter below approximately 10 μm.

16. The solid ranolazine of claim 13, having a particle size distribution wherein approximately 50% of the total volume (D₅₀) is made of particles having a diameter below approximately 40 μm.

17. The solid ranolazine of claim 13, having a particle size distribution wherein approximately 90% of the total volume (D₉₀) is made of particles having a diameter below approximately 80 μm.

18. Solid ranolazine obtained according to the process of claim 2.

19. Solid ranolazine obtained according to the process of claim 3.

20. Solid ranolazine obtained according to the process of claim 12.
